(19) United States
(12) Patent Application Publication
(10) Pub. No.: US 2010/0063407 A1
(43) Pub. Date: Mar. 11, 2010

(54) PORTABLE ELECTROCARDIOGRAM MONITORING DEVICE

(76) Inventor: Lin-Hsiang HUANG, Hsinchu City (TW)

Correspondence Address: SINORICA, LLC, 2275 Research Blvd., Suite 500, ROCKVILLE, MD, 20850, US

(21) Appl. No.: 12/266,067

(22) Filed: Nov. 6, 2008

(30) Foreign Application Priority Data
Sep. 9, 2008 (TW) 097216267

(51) Int. Cl. A61B 5/0402

(52) U.S. Cl. 600/509

(57) ABSTRACT

A portable electrocardiogram monitoring device comprises a case, at least two electrode lead patches, and an internal circuit provided inside the case for receiving a signal from the at least two electrode lead patches. The internal circuit comprises a power supply module and a data transmission module. The power supply module comprises a low-noise voltage stabilizing circuit having an independent first ground plane and a voltage regulation circuit having an independent second ground plane. The data transmission module comprises an operational amplifying circuit having an independent third ground plane and a data processing circuit having an independent fourth ground plane. And, the independent ground planes are connected with one another only by a ground channel.

### FIELD OF THE INVENTION

The present invention relates to a portable electrocardiogram monitoring device, and more particularly to a portable electrocardiogram monitoring device having a low-noise voltage stabilizing circuit, a voltage regulation circuit, an operational amplifying circuit, and a data processing circuit that have independent ground planes respectively and these ground planes are connected with one another only via a ground channel.

### BACKGROUND OF THE INVENTION

The person's electrocardiogram (ECG or EKG) signals have important relation with that person's health conditions, so it is able to understand a person's whole body operational functions from ECG signals. Various kinds of ECG signal measuring devices, such as Electrocardiography (ECG) for medical application, have been researched and developed for monitoring and controlling a person's health conditions via ECG signals, so that a user can know his or her body conditions from the measured ECG signal generated by such measuring device.

A general ECG signal measuring device mainly comprises a plurality of electrode leads, wires, and an ECG signal measuring module. The electrode pieces are attached to a person's body, and the wires are electrically connected between the electrode leads and ECG signal measuring module for transmitting ECG signals detected by the electrode leads to the ECG signal measuring module. The ECG signals measuring module then analyzes the received ECG signals and shows them on a monitor thereof.

In prior art, the internal circuits layout of ECG measuring devices have a common ground plane, that is, the ground plane is not divided for different circuits. As a result, noises generated from the internal and external circuits may couple into ECG signals shown on the monitoring display to adversely affect the accuracy of the ECG signals.

The conventional personal ECG signal measuring devices are supplied along with an AC Power from the wall surface, so that the power can be supplied to the ECG signal measuring devices. However, such personal ECG signal measuring devices could not be used outdoors where no socket is available. Further, conventional ECG signal measuring device does not include a voltage stabilizing circuit to reduce noise from an external power supply.

### SUMMARY OF THE INVENTION

Therefore, one of objects of the present invention is to provide a portable electrocardiogram monitoring device having a low-noise voltage stabilizing circuit, a voltage regulation circuit, an operational amplifying circuit, and a data processing circuit that have the independent ground planes respectively in the circuit layout to prevent noise interference among them.

Another object of the present invention is to provide a portable electrocardiogram monitoring device having a low-noise voltage stabilizing circuit, a voltage regulation circuit, an operational amplifying circuit, and a data processing circuit that have the independent ground planes respectively, and these independent ground planes are connected with one another only via a ground channel so that these independent ground planes can still keep substantially the same voltage reference level.

A further object of the present invention is this portable electrocardiogram monitoring device is functional by having a data bus cable along with DC power source which connecting to an outside computer device. Through the same cable, the measurement data can be transmitted to outside computer device and power source can be also supplied into electrocardiogram monitoring device.

A further object of the present invention is to provide a portable electrocardiogram monitoring device having a low-noise voltage stabilizing circuit to reduce noise effectively from an external power source, to which the portable electrocardiogram monitoring device is connected.

To achieve the above-mentioned objects, the present invention discloses a portable electrocardiogram monitoring device comprising a case, and at least two electrode leads, and an internal circuit. The internal circuit is located inside the case for receiving a signal from the at least two electrode leads. The internal circuit comprises a power supply module and a data transmission module. The power supply module comprises a low-noise voltage stabilizing circuit and a voltage regulation circuit. The low-noise voltage stabilizing circuit has an independent first ground plane, and the voltage regulation circuit has an independent second ground plane. The data transmission module comprises an operational amplifying circuit and a data processing circuit. The operational amplifying circuit has an independent third ground plane, and the data processing circuit has an independent fourth ground plane. The independent first, second, third and fourth ground planes are designed for preventing from noise interference among the circuits. The independent ground planes are connected with one another only by a ground channel for keeping substantially the same voltage reference level.

With the above arrangements, the portable electrocardiogram monitoring device of the present invention has the following advantages:
(1) Each of the low-noise voltage stabilizing circuit, the voltage regulation circuit, the operational amplifying circuit, and the data processing circuit has an independent ground plane, so that noise interference among these circuits can be prevented effectively.
(2) The independent ground planes are connected with one another only by a ground channel, so as to keep substantially the same voltage reference level.
(3) The portable electrocardiogram monitoring device can be connected to an external power supply via a data bus cable provided on the case for directly utilizing 5V voltage source provided by the external power supply.
(4) The low-noise voltage stabilizing circuit reduces noise effectively from coupling into data signal of the portable electrocardiogram monitoring device.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view of a portable electrocardiogram monitoring device according to the present invention;

FIG. 2 is a block diagram of an internal circuit for the portable electrocardiogram monitoring device according to a first embodiment of the present invention;

FIG. 3 is a block diagram of an internal circuit for the portable electrocardiogram monitoring device according to a second embodiment of the present invention;

FIG. 4 is an electrocardiogram shown on a monitoring display when the portable electrocardiogram monitoring device of the present invention is provided with a digital filter; and

FIG. 5 is an electrocardiogram shown on a monitoring display when the portable electrocardiogram monitoring device of the present invention is not provided with a digital filter.

### DETAILED DESCRIPTION OF THE PREFERRED EMBODIMENTS

Exemplary embodiments of the present invention are described herein in the context of a portable electrocardiogram monitoring device.

Those of ordinary skilled in the art will realize that the following detailed description of the exemplary embodiment(s) is illustrative only and is not intended to be in any way limiting. Other embodiments will readily suggest themselves to such skilled persons having the benefit of this disclosure. Reference will now be made in detail to implementations of the exemplary embodiment(s) as illustrated in the accompanying drawings. The same reference indicators will be used throughout the drawings and the following detailed description to refer to the same or like parts.

In accordance with the embodiment(s) of the present invention, the components, process steps, and/or data structures described herein may be implemented using various types of operating systems, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Erasable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card and paper tape, and the like) and other known types of program memory.

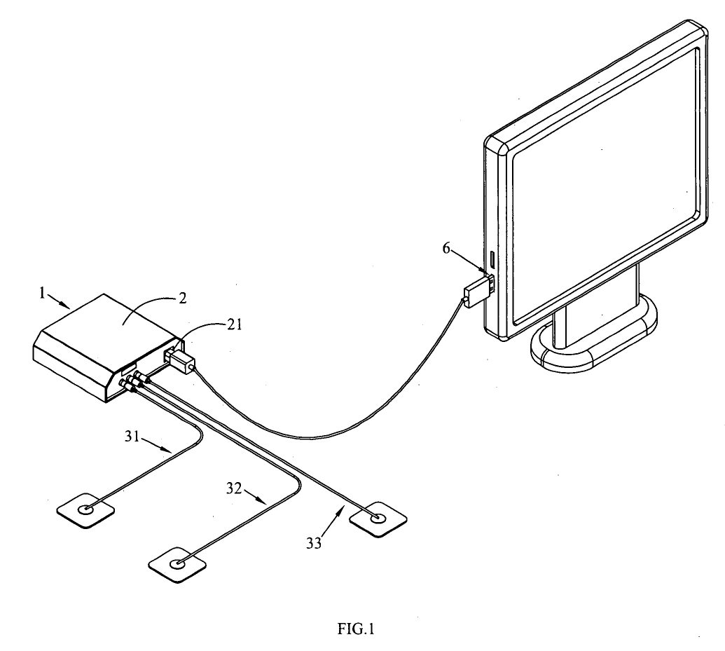

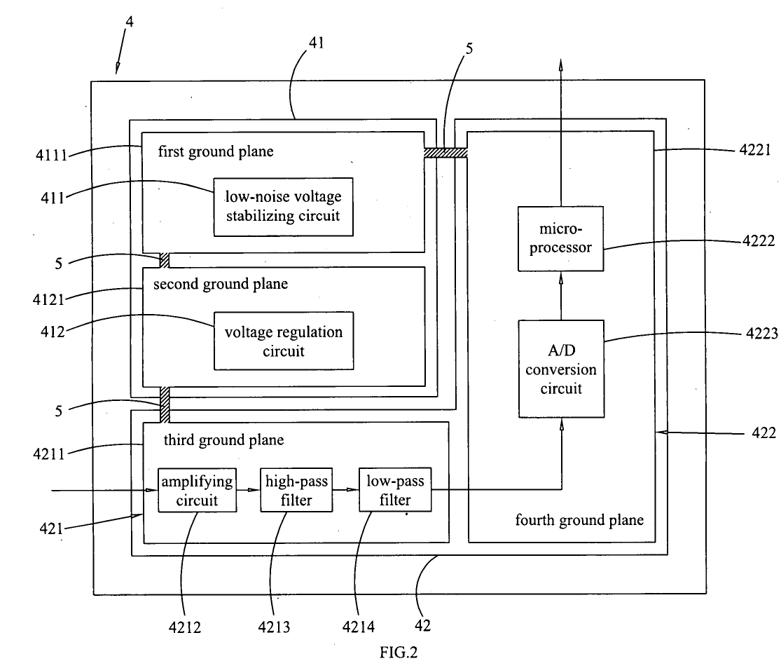

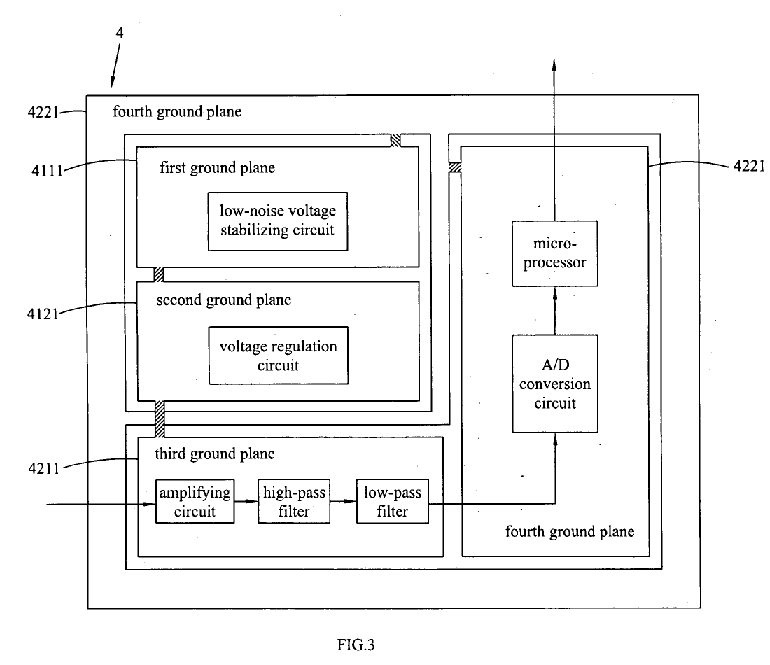

Please refer to FIG. 1 that is a perspective view of a portable electrocardiogram monitoring device according to the present invention. As shown, the portable electrocardiogram monitoring device 1 comprises a case 2, a plurality of electrocardiogram (ECG) electrode lead patches, and an internal circuit 4. The case 2 is installed with a data bus cable 21, which can be electrically connected to a power supply module 41 and a data transmission module 42 of the internal circuit 4 (shown in FIGS. 2 and 3). Preferably, the data bus cable 21 can be a cable with a universal serial bus (USB) connector, a mini-USB connector, or a micro-USB connector. While the data bus cable 21 is connected to an external data bus port 6 of an electronic device, the portable electrocardiogram monitoring device 1 can receive the power supplied from the external device via the same cable comprising with the data bus and DC power source.

Each ECG electrode lead patch 31 comprises an electrode patch and a wire. The electrode patch is connected at an end to the wire. In this embodiment, the portable electrocardiogram monitoring device 1 is illustrated by using three ECG lead electrode patch, namely, a first ECG electrode lead patch 31, a second ECG lead electrode patch 32, and a third ECG electrode lead patch 33. However, it is understood the number of the ECG electrode lead patches is not restricted to three. Two of the three ECG electrode lead patches are used for transmitting an analog signal to the internal circuit 4.

FIG. 2 illustrates a block diagram of an internal circuit for the portable electrocardiogram monitoring device according to a first embodiment of the present invention. As shown, the internal circuit 4 is located inside the case 2 for receiving the analog signal from at least two of the plurality of ECG electrode lead patches, and comprises a power supply module 41 and a data transmission module 42.

The power supply module 41 is capable of receiving an external power via the data bus cable 21, so as to supply power to the data transmission module 42. The power supply module 41 comprises a low-noise voltage stabilizing circuit 411 and a voltage regulation circuit 412. The low-noise voltage stabilizing circuit 411 can reduce the noise effectively from the internal and external circuits, and has an independent first ground plane 4111. The voltage regulation circuit 412 converts a 5V power supplied from the external data bus port 6 into symmetric positive and negative direct current (DC) voltage sources which is provided to the operational amplifying circuit 421 of the data transmission module 42. The voltage regulation circuit 412 has an independent second ground plane 4121.

The data transmission module 42 transmits the analog signal provided by two ECG electrode lead patches, and comprises an operational amplifying circuit 421 and a data processing circuit 422. The operational amplifying circuit 421 has an independent third ground plane 4211, an amplifying circuit 4212, a high-pass filter 4213, and low-pass filter 4214. The amplifying circuit 4212 amplifies the received analog signal; the high-pass filter 4213 filters out signals having a frequency lower than a cutoff frequency; and the low-pass filter 4214 filters out signals having a frequency higher than a cutoff frequency.

The data processing circuit 422 has an independent fourth ground plane 4221, a microprocessor 4222, and an analog-to-digital (A/D) conversion circuit 4223. The A/D conversion circuit 4223 converts the analog signal into a digital signal. The first ground plane 4111, the second ground plane 4121, the third ground plane 4211, and the fourth ground plane 4221 are independent of one another to thereby effectively prevent noise interference among different circuits in the case 2. It should be noted that the first ground plane 4111, the second ground plane 4121, the third ground plane 4211, and the fourth ground plane 4221 in the circuit layout are connected with one another only by a ground channel 5. Preferably, a ratio of the area of each of the ground channels 5 to the area of each of the first, the second, the third, and the fourth ground plane 4111, 4121, 4211, 4221 is ranged between 1:10 and 1:100000. In the illustrated embodiments of the present invention, the third ground plane 4211 and the second ground plane 4121 are connected with each other via one ground channel 5, the second ground plane 4121 and the first ground plane 4111 are connected with each other via one ground channel 5, and the first ground plane 4111 and the fourth ground plane 4221 are connected with each other via one ground channel 5. With these arrangements, all the ground planes 4111, 4121, 4211, and 4221 can still keep substantially the same voltage reference level. The analog signal provided by two ECG electrode lead patches is amplified by the operational amplifying circuit 421 and then sent to the data processing circuit 422 to be converted into a digital signal as required by an electrocardiogram. That is, the digital signal is an ECG signal.

FIG. 3 illustrates a block diagram of an internal circuit for the portable electrocardiogram monitoring device according to a second embodiment of the present invention. As shown, the difference between the second embodiment and the first embodiment is that the area of the fourth ground plane 4221 of the second embodiment is different form that of the first embodiment. In the second embodiment, the independent fourth ground plane 4221 is connected via one ground channel 5 to an external area surrounding the first ground plane 4111, the second ground plane 4121, the third ground plane 4211, and the fourth ground plane 4221. Therefore, the fourth ground plane 4221 in the second embodiment comprises the external area surrounding the first ground plane 4111, the second ground plane 4121, the third ground plane 4211, and the fourth ground plane of the first embodiment. By connecting the first ground plane 4111 to the external area surrounding the fourth ground plane via a ground channel 5, the first, the second, the third, and the fourth ground plane 4111, 4121, 4211, and 4221 can still keep the same voltage reference level. Therefore, parts that are the same in the two embodiments are not repeatedly described herein.

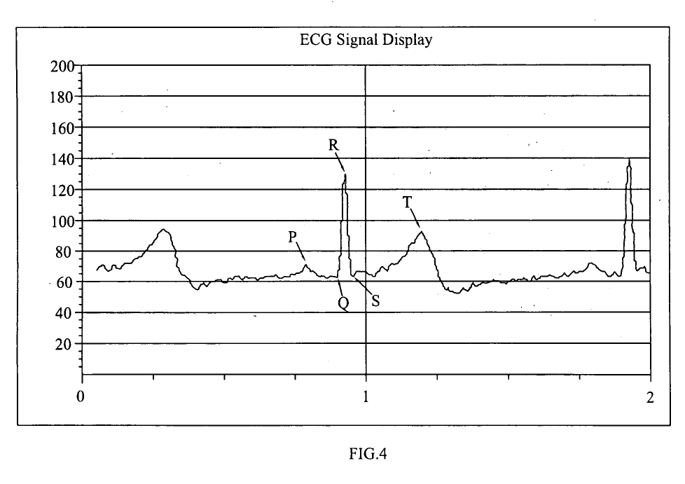

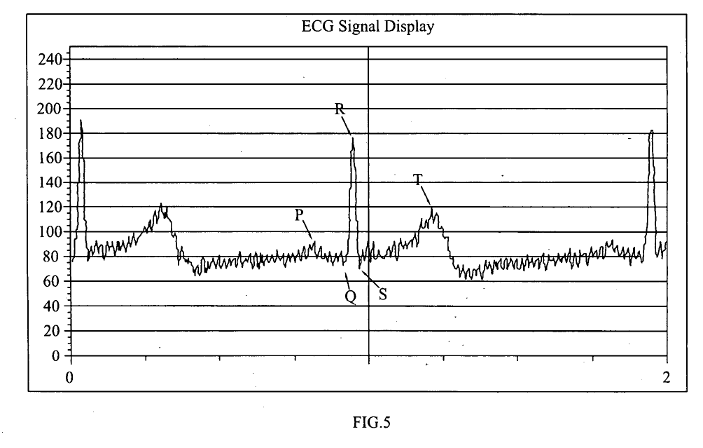

FIGS. 4 and 5 illustrate the examples of electrocardiograms shown on a monitoring display when the portable electrocardiogram monitoring device of the present invention is provided with and not provided with a digital filter, respectively. The digital filter module is implemented as software for filtering out noise of a digital signal. By using the digital filter module which is installed on the external computer and receives the digital signal output by the A/D conversion circuit 4223, some wave points on the electrocardiogram that must be observed, such as P wave point, Q wave point, R wave point, S wave point and T wave point can be clearly seen on a monitor of the external computer, as shown in FIG. 4. On the other hand, when no digital filter module is installed on the external computer, after the digital signal is inputted to the external computer, the digital signal is subject to noise interference from the external computer, and the electrocardiogram shown on the monitoring display of the external computer has complicate waveforms, making it difficult to clearly observe the P wave point, Q wave point, R wave point, S wave point, and T wave point on the electrocardiogram.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A portable electrocardiogram monitoring device, comprising:
at least two electrode lead patches; and
an internal circuit capable of receiving an analog signal provided by the at least two electrode lead patches, and the internal circuit consisting of:
a power supply module including a low-noise voltage stabilizing circuit and a voltage regulation circuit, and the low-noise voltage stabilizing circuit having an independent first ground plane, and the voltage regulation circuit having an independent second ground plane; and
a data transmission module including an operational amplifying circuit and a data processing circuit, and the operational amplifying circuit having an independent third ground plane, and the data processing circuit having an independent fourth ground plane, wherein the power supply module supplies power to the data transmission module, so that the analog signal is amplified by the operational amplifying circuit and transmitted to the data processing circuit to generate a digital signal as required by an electrocardiogram;
wherein the independent first, second, third, and fourth ground planes are connected with one other only by a ground channel.

2. The portable electrocardiogram monitoring device as claimed in claim 1, wherein the operational amplifying circuit comprises a high-pass filter for filtering out signals having a frequency lower than a cutoff frequency.

3. The portable electrocardiogram monitoring device as claimed in claim 1, wherein the operational amplifying circuit comprises a low-pass filter for filtering out signals having a frequency higher than a cutoff frequency.

4. The portable electrocardiogram monitoring device as claimed in claim 1, further comprising a data bus cable being connected to the outside computing device, so that measurement data can be transmitted to computing device and the external power can be also supplied into electrocardiogram monitoring device via the same data bus cable.

5. The portable electrocardiogram monitoring device as claimed in claim 1, wherein the data processing circuit comprises a microprocessor.

6. The portable electrocardiogram monitoring device as claimed in claim 1, wherein the data processing circuit comprises an A/D conversion circuit for converting the analog signal into the digital signal.

7. The portable electrocardiogram monitoring device as claimed in claim 1, further comprising a digital filter module for filtering out noise from the digital signal.

8. The portable electrocardiogram monitoring device as claimed in claim 1, wherein the voltage regulation circuit converts an external 5V power supply into symmetric positive and negative direct current (DC) voltage sources.

9. The portable electrocardiogram monitoring device as claimed in claim 1, wherein each of the electrode lead patches has an end connected to a wire.

10. The portable electrocardiogram monitoring device as claimed in claim 1, wherein a ratio of an area of each of the ground channels to an area of each of the ground planes is ranged from 1:10 to 1:100000.
